(19) United States
(12) Patent Application Publication
(10) Pub. No.: US 2002/0009424 A1
(43) Pub. Date: Jan. 24, 2002

(54) Foamed fragrancing and deodorizing gel

(76) Inventors: Thomas V. Kandathil, Racine, WI (US); Ryuji Kawamoto, Kanagawa (JP); Maciej K. Tasz, Racine, WI (US); John H. Engelman, Racine, WI (US); Roberta R. Wu, Chicago, IL (US)

Correspondence Address: S.C. JOHNSON & SON, INC., 1525 HOWE STREET, RACINE, WI, 53403-2236, US

(21) Appl. No.: 09/758,704

(22) Filed: Jan. 11, 2001

Related U.S. Application Data
(60) Provisional application No. 60/176,214, filed on Jan. 14, 2000.

(51) Int. Cl. A61K 7/075; A61K 7/08; A61K 7/50; C11D 17/00

(52) U.S. Cl. 424/70.22; 424/70.27; 510/152

(57) ABSTRACT

Disclosed herein are foamed gel air-treating compositions that have at least 50% water, between 1 and 15 percent soap surfactant, a volatile fragrance, at least 0.1 percent carbon, and a foam stabilizer. These compositions can fragrance a room as well as adsorb malodors in the room. Methods for forming these foamed gels are also disclosed.

### CROSS REFERENCES TO RELATED APPLICATIONS

This application claims the benefit of U.S. Provisional Application No. 60/176,214 filed Jan. 14, 2000.

### STATEMENT REGARDING FEDERALLY SPONSORED RESEARCH

Not applicable.

### BACKGROUND OF THE INVENTION

The present invention relates to air treating gels of the type that dispense a volatile fragrance without the need for an external heating source.

The problem of malodors has been with mankind for as long as civilization. There are very few articles which do not have some odor associated with them. Often this odor will change over time and either the original or the new odor is perceived as a malodor.

Burnable candles and incense sticks dispense fragrances to mask such malodors. Other systems rely on electrical or gas heaters to externally supply heat to cause the fragrance to disperse from a gel or other substrate. See e.g. U.S. Pat. No. 5,928,605. The disclosure of this patent, and all other patents referred to herein, are incorporated by reference as if fully set forth herein.

For some applications it is desirable to have a system where the perfume/fragrance disperses in a controlled manner without the need for external heat or burning of the object itself. A variety of gels were developed in which perfume was temporarily, but not permanently, trapped. See e.g. U.S. Pat. No. 4,178,264.

It is has also been proposed to form fragrancing gels as a foam in order to increase their volume and reduce the trapping of the fragrance. See U.S. Pat. No. 4,755,377.

In any event, systems that focus on the vaporization of the fragrance are typically not well suited to absorb the malodor from the air. They are much more concerned with masking the malodor than removing it. As a result, such systems often use strong fragrances (and/or require greater concentrations of fragrance) to be effective.

U.S. Pat. No. 4,294,821 discloses that sodium bicarbonate, activated charcoal, zinc ricinoleate, certain glycols such as ethylene, and certain other compositions can be used to absorb a malodor from the air. However, such systems often do not pull sufficient malodor out of the air, absorb the malodor too slowly, allow re-releasing the malodor from the substrate once it had been absorbed, or are otherwise deficient.

There has also been an attempt to combine a gel fragrancer with an odor absorber. This involved including activated carbon in a fragrancer gel. However, the density of the base material in this gel raised the cost per unit volume of the product and the product had decreased volatility.

It can therefore be seen that a need exists for an improved gel type fragrancer.

### BRIEF SUMMARY OF THE INVENTION

In one aspect the invention provides a foamed gel air-treating composition. It has at least 50% by weight water, between 1 and 15 percent by weight soap surfactant, a volatile fragrance, and at least 0.1 percent by weight carbon (as elemental carbon). There can also be a surfactant foam stabilizer.

The water is preferably deionized water in the 70-90 percent weight range (more preferably in the 80-90 percent weight range).

The soap surfactants are reaction products of a fatty acid or fatty acid ester with an alkali (sodium or potassium hydroxide). Fatty acids are those with four to twenty-two carbons (and/or mixtures thereof). The most preferred fatty acids for forming such soaps are C₁₂-C₂₀, with sodium stearate being the soap of choice.

A wide variety of volatile fragrances/perfumes can be included in these gels such as essential oils obtained from leaves, flowers, fruits, roots, bark, seeds, and wood. Examples are oil of rose, oil of lime, oil of pine, oil of wintergreen, oil of lemon grass, oil of cinnamon, oil of almond and oil of sandlewood. Synthetically derived fragrancing materials, for example alcohols, esters, aldehydes and ketones, may also be used as the perfume constituent, typically in a mixture with the natural substances. Synthetically obtained compounds also may include the synthetically prepared odoriferous active materials of the essential oils. The perfume constituent is frequently a complex mixture essential oils and/or synthetic fragrancing materials.

The carbon is elemental carbon. The preferred forms are coke, charcoal and activated charcoal. It is in any event highly desirable that at least some of the carbon (preferably a majority of the carbon) is exposed on the outer surface of the fragrancer.

To prevent the carbon and other components from destabilizing the foam, inclusion of some form of foam stabilizer is preferred. While a variety of nonionic, anionic, cationic, or amphoteric surfactants are of some benefit in this regard, particularly beneficial are foam stabilizers which are nitrogen containing, particularly those which are amine oxides, amids, or betaines (e.g. C₈-C₂₀ betaines such as those with carboxylate or sulfate anions). Lauramine oxide (available from Stepan Chemical as Ammonyx LO) and 101-CG (available from Rhone-Poulenc) have been selected for our experiments.

To avoid syneresis one can add polyvinyl alcohol or pyrrolidone.

In another aspect the invention provides methods for forming a bulk of the above compositions. One stirs the water, soap surfactant, fragrance, and foam stabilizer at a temperature sufficient to form a soap solution, then stirs them at a temperature between 28 C. and 43 C. Thereafter, one allows the composition to form the bulk and adds the carbon (e.g. on the outside surface). The mixing in the 28 C.-43 C. range permits foam that forms to be stable. The mixing process can be facilitated by glycol or another material that helps to form a gel/foam matrix.

The invention also provides a method for fragrancing and at least partially deodorizing room air. One exposes air in a room to a bulk of the composition and allows the fragrance to disperse into the air. Simultaneously, the malodor binds to the bulk.

It has been discovered that in these foamed gels the carbon can act to bind malodors without significantly interfering with foam formation, stability, or the ability of the fragrance to volatize. This effect is most efficient when most of the carbon is exposed to the air at the surface of the bulk.

The advantages of the present invention include providing compositions of the above kind:

(a) that appear to consumer to have sufficient size, yet which use reduced levels of materials;

(b) which both deodorize and fragrance a room, without the need for an external heat or energy supply; and

(c) which have relatively low cost.

These and still other advantages of the present invention (e.g. providing methods for making and using such compositions) will be apparent from the description which follows. The following description is merely of the preferred embodiments. The claims should be looked to in order to understand the full scope of the invention.

### DETAILED DESCRIPTION OF THE INVENTION

### Preferred Method Of Making

Batches of the compositions were made using the following protocol. We first heated water, propylene glycol, sodium hydroxide and foam stabilizing surfactant to 62-66 C., and used moderate to slow stirring (300-400 RPM) to blend them. We then added the stearic acid with stirring, and allowed the composition to stir and react for 15-20 minutes. The resulting mixture was clear with no pieces of stearic acid.

With vigorous stirring (600-800 RPM) we next added the dye. After about 5 minutes the composition was even in color. The composition was then removed from the heat, and water was added to compensate for water lost through evaporation.

We then returned the composition to the heat and brought back the temperature to 62 C. We then removed the composition from the heat, added fragrance and continued stirring vigorously. When the composition had dropped to about 59 C., we increased stirring to 1000 RPM. When the temperature had dropped to about 56 C., we poured the composition into containers, and to generate significant foam we continued to stir until the temperature had dropped to about 32 C. We then added the activated carbon as the samples were skinning over.

### Formulations

The following formulations of the present invention were prepared pursuant to the above protocol.

| Ingredient | R72-1 | R72-2 | R72-3 | R72-4 | R72-5 | 1006 |
|---|---|---|---|---|---|---|
| Deionized water | 83.2494 | 82.7494 | 86.2494 | 83.9394 | 80.0394 | 82.6860 |
| Propylene glycol | 3.0000 | 3.0000 | 0.0000 | 1.0000 | 3.0000 | 0.0000 |
| Sodium hydroxide (50%) | 1.7500 | 1.7500 | 1.7500 | 2.0600 | 2.0600 | 2.3040 |
| Ammonyx LO | 0.0000 | 0.0000 | 0.0000 | 0.0000 | 1.8000 | 0.0000 |
| Rhone Poulenc 101-CG | 0.0000 | 0.5000 | 0.0000 | 0.0000 | 0.0000 | 0.0000 |
| Stearic acid 67% | 6.0000 | 6.0000 | 6.0000 | 7.0000 | 7.0000 | 8.0000 |
| Perfume | 4.0000 | 4.0000 | 4.0000 | 4.0000 | 4.0000 | 4.0000 |
| Kathon CG-ICP | 0.0000 | 0.0000 | 0.0000 | 0.0000 | 0.1000 | 0.0000 |
| Activated carbon Vapure 612 | 2.0000 | 2.0000 | 2.0000 | 2.0000 | 2.0000 | 2.0000 |
| Dye | 0.0006 | 0.0006 | 0.0006 | 0.0006 | 0.0006 | 0.0100 |
| Polyvinyl alcohol | 0.0000 | 0.0000 | 0.0000 | 0.0000 | 0.0000 | 1.000 |
| Total | 100.00 | 100.00 | 100.00 | 100.00 | 100.00 | 100.00 |

### Testing of the Compounds

Compounds of the present invention were tested by a panel of consumers as against a competitive passive gel fragrancer (with usage in a manner similar to the way the competitive product was typically used). Respondents were given a product to try for four weeks. Bulk amounts of the product were placed in a room. After the four weeks they were visited by interviewers and questioned regarding the attributes of the product. The present invention was rated as superior in odor removal.

It should be appreciated that the invention provides a way of more efficiently dispersing fragrance without the need for electrical heaters or burners. Not only is malodor masked, some of it is also removed. For a given size product, less material needs to be used.

The invention is not to be limited to the specific embodiments described above. Other modifications are intended to be within the scope of the invention. For example, in place of propylene glycol one could use mono, di, tri, and poly hydric alcohols and glycols as processing aids.

Further, antimicrobial agents can be used. Also, hardening and/or weight agents such as calcium carbonate, calcium sulfate, magnesium carbonate, talc, synthetic and natural clays, silica and borax can be included. Natural gelling agents such as guar gum, gum arabic, locust bean gum, carrageenan gum, and synthetic gelling agents cellulose such as carboxymethyl cellulose, hydroxymethyl and propyl cellulose and Kelco Gel can be used. Synthetic clays such as laponte and optigel can also be used. Thus, the claims should be looked to in order to judge the full scope of the invention.

### Industrial Applicability

The invention provides compositions for fragrancing and deodorizing air, methods of making the compositions, and methods of using them.

We claim:

1. A foamed gel air-treating composition, comprising:
at least 50% by weight water;
between 1 and 15 percent by weight soap surfactant;
a volatile fragrance; and
at least 0.1 percent by weight carbon as carbon.

2. The foam gel air-treating composition of claim 1, further comprising a foam stabilizer.

3. The foam gel air-treating composition of claim 2, wherein the foam stabilizer is a nitrogen containing surfactant.

4. The foam gel air-treating composition of claim 3, wherein the foam stabilizer is selected from the group consisting of amine oxides, amids and betaines.

5. The foam gel air-treating composition of claim 1, wherein the soap surfactant is a stearate salt.

6. The foam gel air-treating composition of claim 1, wherein the carbon is selected from the group consisting of charcoal, activated charcoal and coke.

7. The foam gel air-treating composition of claim 1, wherein the composition is in the form of a bulk, and a majority of the carbon is on an outside surface of the bulk.

8. The foam gel air-treating composition of claim 1, further comprising polyvinyl alcohol.

9. A method for forming a bulk of the composition of claim 2, comprising:
stirring the water, soap surfactant, fragrance, and foam stabilizer at a temperature sufficient to provide a soap solution;
then stirring the water, soap surfactant, fragrance, and foam stabilizer at a temperature between 28 C. and 43 C. to generate a foam gel matrix; and
thereafter allowing the composition to form a bulk and adding carbon to at least an outer exposed surface of the bulk.

10. A method for fragrancing and at least partially deodorizing air in a room having a malodor, comprising:
exposing the air in the room to a bulk of the claim 1 composition and allowing fragrance to disperse from the bulk to the air and allow a portion of the malodor to bind to the bulk.
